(19) United States
(12) Patent Application Publication
(10) Pub. No.: US 2023/0189862 A1
(43) Pub. Date: Jun. 22, 2023

(54) EDIBLE FUNGI

(71) Applicant: MARLOW FOODS LIMITED, Stokesley (GB)

(72) Inventors: OLUMUYIWA A. AKINTOYE, Stokesley Yorkshire (GB); Alexander James EVANS, Stokesley Yorkshire (GB); Jonathan Paul MILLER, Stokesley Yorkshire (GB)

(21) Appl. No.: 17/926,940

(22) PCT Filed: May 14, 2021
(86) PCT No.: PCT/GB2021/051162

§ 371 Date: Nov. 21, 2022

(30) Foreign Application Priority Data
May 22, 2020 (GB) 2007677.4

(51) Int. Cl. A23L 31/00; A23C 11/10; A23L 33/175

(52) U.S. Cl. CPC A23L 31/00; A23C 11/10; A23L 33/175

(57) ABSTRACT

A milk-like aqueous formulation which does not require high levels of flavourants and/or sugar but comprises high levels of amino acids and/or proteins is described. The aqueous formulation comprising fungal particles of a filamentous fungus, wherein the particles include relatively low levels of acetyl-glutamic acid, uridine, inosine, guanosine, UMP, GMP, AMP, sodium, potassium and/or ammonium.

This invention relates to edible fungi and particularly, although not exclusively, relates to foodstuffs comprising edible fungi. Preferred embodiments relate to milk-like aqueous formulations.

Dairy-based milks are of course very widely consumed, either as drinks or as ingredients in other foodstuffs. However, such milks are not acceptable to all consumers, for example vegans or consumers intolerant to ingredients in the milks, such as lactose.

Non-dairy based milks are known, such as soy milk, almond milk or rice milk.

There have been proposals to formulate milks which comprise edible fungi. However, edible fungi tend to have a mushroom like and/or savoury taste. Such undesirable tastes need to be masked or eliminated in any milks containing fungi, for example by means of high levels of flavourants and/or sugars. The high levels which need to be used are undesirable, particularly if the milk is intended to be marketed as having health benefits. However, prior hereto, it has proved difficult to mask the undesirable taste in an acceptable manner. As a result, any fungi-based milks may have low consumer acceptability.

Edible fungi, for example Fusarium venenatum, has been shown to be rich in essential amino acids which may provide a greater anabolic response compared to cow’s milk (see Dunlop et al. Br J Nutr. 2017; 11:1-13). Applicant believes it is desirable for any milk incorporating edible fungi to be rich in the essential amino acids, whilst addressing problems associated with the mushroom taste described.

It is an object of the present invention to address the above-described problems.

It is an object of the present invention to provide a milk-like aqueous formulation which does not require high levels of flavourants and/or sugar.

It is an object of the present invention to provide a milk-like aqueous formulations comprising edible fungi which includes high levels of amino acids and/or proteins.

According to a first aspect of the invention, there is provided an aqueous formulation comprising fungal particles of a filamentous fungus.

Unless otherwise specified “parts per million” or “ppm” refers to the number of units of mass of a specified component per million units of a total specified mass. Thus, ppm is quoted on a weight for weight basis.

A reference to characteristics on a wet matter basis suitably means that the amount of water included in a specified mass is taken into consideration in calculating the ppm. A reference to characteristics on a dry matter basis suitably means that the amount of water included in a mass is ignored in calculating the ppm.

The fungal particles preferably collectively include at least one of the following parts per million of a specified component described in (a) to (j), relative to the total mass of the fungal particles, on a dry matter basis:
(a) less than 3 ppm of acetyl-glutamic acid;
(b) less than 160 ppm of uridine;
(c) less than 75 ppm of inosine;
(d) less than 625 ppm of guanosine;
(e) less than 660 ppm of UMP;
(f) less than 800 ppm of GMP;
(g) less than 875 ppm of AMP;
(h) less than 330 ppm of sodium;
(i) less than 2300 ppm of potassium;
(j) less than 1500 ppm of ammonium.

Characteristics and/or components described herein may be assessed as described in the examples.

Said fungal particles may include at least two of the characteristics (a) to (j). Said fungal particles may include at least six of the characteristics (a) to (j). Said fungal particles may include all of the characteristics (a) to (j). Said fungal particles may include any of characteristics (e) to (g).

In a first preferred embodiment, the fungal particles preferably collectively include at least one of the following parts per million of a specified component described in (k) to (t), relative to the total mass of the fungal particles, on a dry matter basis:
(k) less than 2 ppm of acetyl-glutamic acid;
(I) less than 80 ppm of uridine;
(m) less than 50 ppm of inosine;
(n) less than 400 ppm of guanosine;
(o) less than 400 ppm of UMP;
(p) less than 400 ppm of GMP;
(q) less than 400 ppm of AMP;
(r) less than 330 ppm of sodium;
(s) less than 1000 ppm of potassium;
(t) less than 1000 ppm of ammonium.

Said fungal particles may include at least two of the characteristics (k) to (t). Said fungal particles may include at least six of the characteristics (k) to (t). Said fungal particles may include all of the characteristics (k) to (t). Said fungal particles may include any of characteristics (o) to (q).

In a second preferred embodiment, the fungal particles preferably collectively include at least one of the following parts per million of a specified component described in (u) to (dd), relative to the total mass of the fungal particles, on a dry matter basis:
(u) less than 1 ppm of acetyl-glutamic acid;
(v) less than 42ppmof uridine;
(w) less than 40 ppm of inosine;
(x) less than 120 ppm of guanosine;
(y) less than 160 ppm of UMP;
(z) less than 200 ppm of GMP;
(aa) less than 200 ppm of AMP;
(bb) less than 300 ppm of sodium;
(cc) less than 1000 ppm of potassium;
(dd) less than 1000 ppm of ammonium.

Said fungal particles may include at least two of the characteristics (u) to (dd). Said fungal particles may include at least six of the characteristics (u) to (dd). Said fungal particles may include all of the characteristics (u) to (dd). Said fungal particles may include any of characteristics (y) to (aa).

Said fungal particles preferably includes at least one of the following characteristics, wherein an amount of a component is specified per 100 g of said fungal particles on a dry matter basis:
(A) at least 4.1 g/1 00 g of aspartic acid;
(B) at least 2.0 g/100 g of serine;
(C) at least 6.0 g/100 g of glutamic acid;
(D) at least 2.0 g/100 g of glycine;
(E) at least 1.0 g/100 g of histidine;
(F) at least 2.8 g/100 g of arginine;
(G) at least 2.0 g/100 g of threonine;
(H) at least 0.6 g/100 g of alanine;
(I) at least 2.4 g/100 g of proline;
(J) at least 0.2 g/100 g of cystine;
(K) at least 1.2 g/100 g of tyrosine;
(L) at least 2.8 g/100 g of valine;
(M) at least 0.8 g/100 g of methionine;
(N) at least 4.0 g/100 g of lysine;
(O) at least 2.0 g/100 g of iso-leucine;
(P) at least 3.2 g/100 g of leucine
(Q) at least 1.6 g/100 g of phenylalanine;
(R) at least 0.6 g/1 00 g of tryptophan.

Said fungal particles may include at least five, preferably at least ten, more preferably at least fifteen of characteristics (A) to (R).

Said fungal particles suitably are of a filamentous fungus (herein also referred to as “fungal particles”). Said filamentous fungus preferably comprises fungal mycelia and suitably at least 80 wt%, preferably at least 90 wt%, more preferably at least 95 wt% and, especially, at least 99 wt% of the fungal particles in said mass comprise fungal mycelia. Some filamentous fungi may include both fungal mycelia and fruiting bodies. Said fungal particles preferably comprise a filamentous fungus of a type which does not produce fruiting bodies. Where, however, a filamentous fungus of a type which produces fruiting bodies is used, the fungal particles in said mass suitably include at least 80 wt%, preferably at least 90 wt%, more preferably at least 95 wt% of fungal mycelia. Preferably, said fungal particles comprise substantially only fungal mycelia - that is, said fungal particles in said mass preferably do not include any fruiting bodies.

Preferred fungi for said fungal particles have a cell wall which includes chitin and/or chitosan. Preferred fungi have a cell wall which includes polymeric glucosamine. Preferred fungi have a cell wall which includes β1-3 and 1-6 glucans.

Said fungal particles preferably comprise (preferably consist essentially of) fungus, for example selected from fungi imperfecti.

Preferably, said fungal particles comprise, and preferably consist essentially of, cells of Fusarium species, especially of Fusarium venenatum A3/5 (formerly classified as Fusarium graminearum) (IMI 145425; ATCC PTA-2684 deposited with the American Type Culture Collection, 10801 University Boulevard, Manassas, VA.) as described for example in WO96/21361 (Zeneca) and WO95/23843 (Zeneca).

Preferably, said fungal particles are non-viable. Preferably, said fungal particles have been treated to lower the level of RNA which they contain. Thus, the level of RNA in the fungal particles used is preferably less than the level in an identical fungus when in a viable state.

The level of RNA in the fungal particles is preferably less than 2 wt% on a dry matter basis.

Said fungal particles in said aqueous formulation may have a dimension in a first direction of less than 200 µm, wherein said dimension suitably refers to the length of the fungal particles (especially where the fungi are filamentous). The number average length of said fungal particles in said aqueous formulation is suitably less than 250 µm, is preferably less than 100 µm.

The number average of said first dimensions may be at least 1 µm, preferably at least 5 µm, more preferably at least 10 µm.

Suitably, the mean of said dimensions in said first direction is less than 200 µm, preferably less than 100 µm, with a standard deviation on the mean of less than 200 µm, preferably less than 100 µm. The mean is preferably at least 10 µm.

Said fungal particles in said aqueous formulation may have a dimension in a second direction, measured perpendicular to said first direction, which is suitably less than 20 µm, preferably less than 10 µm, more preferably less than 7 µm and especially 5 µm or less. Said dimension in said second direction is preferably at least 1 µm, more preferably at least 3 µm. Said dimension in said second direction is preferably a diameter of the particles and is preferably substantially the same as a dimension in a third direction, perpendicular to the dimension in said second direction. Thus, preferably said particles have a substantially circular cross-section. Preferably, values for the number average of said diameters of said fungal particles in said mass are also as stated above.

Said aqueous formulation is suitably homogenous. The aqueous formulation is suitably flowable. It is preferably a liquid. The viscosity of said aqueous formulation at 0.1 Pa Shear Stress and 10° C. may be at least 0.01 Pa.s, preferably at least 0.1 Pa.s. It may be less than 4.00 Pa.s or less than 1.00 Pa.s.

Said aqueous formulation may comprise at least 5 wt%, suitably at least 10 wt%, preferably at least 15 wt%, especially at least 17.5 wt% of said fungal particles on a dry matter basis. Said aqueous formulation may comprise less than 95 wt%, less than 90 wt% or less than 78 wt% of water. Said aqueous formulation may comprise at least 80 wt%, suitably at least 85 wt% water.

In said aqueous formulation, the ratio defined as wt% of water divided by the wt% of fungal particles (said fungal particles being on a dry matter basis) may be in the range 3 to 6, preferably in the range 4 to 5.

In said aqueous formulation, the sum of the wt% of fungal particles and water is suitably at least 90 wt%, preferably at least 95 wt%, more preferably at least 99 wt%.

Said aqueous formulation is suitably a liquid foodstuff for human consumption. It is preferably a milk. It is preferably a drink. It is preferably substantially white in colour.

Said aqueous formulation preferably incudes 0 wt% of animal milk and 0 wt% of ingredients derived from animal milk. Said aqueous formulation preferably incudes 0 wt% of ingredients derived from an animal source.

Said aqueous formulation may incorporate other ingredients, for example one or more flavouring materials. The sum of the wt% of salts, sugars and flavouring materials in said aqueous formulation is preferably less than 1 wt%, more preferably less than 0.5 wt% or less than 0.2 wt%.

Said aqueous formulation may include a milk flavour additive, suitably at a level of less than 0.1 wt%, preferably less than 0.05 wt%.

The sum of the wt% of all additives in said aqueous formulation over and above the fungal particles and water may be in the range 0.01 to 1 wt%, for example 0.02 to 0.7 wt%.

According to a second aspect of the invention, there is provided a method of making an aqueous formulation according to the first aspect, the method comprising:
(a) selecting an edible mass comprising fungal particles of a filamentous fungus as described in said first aspect; and
(b) contacting said mass with one or more other ingredients.

Said edible mass comprising fungal particles of a filamentous fungus as described in said first aspect may be made in a method comprising:
(i) selecting a precursor mass comprising fungal particles of a filamentous fungus;
(ii) contacting said precursor mass with an aqueous solvent to produce a mixture;
(iii) filtering said mixture;
(iv) isolating a residue which comprises a said edible mass comprising fungal particles of a filamentous fungus.

It is found that the aqueous solvent extracts selected components from the precursor mass, including, advantageously, components responsible for the savoury and/or mushroom like taste of the filamentous fungus. In addition, advantageously, the level of amino acids/protein in said filamentous fungus is not significantly reduced by the process which means that the process importantly does not significantly diminish the nutritional value of the edible mass produced.

In step (ii), preferably, said precursor mass and aqueous solvent are agitated, suitably to intimately mix the precursor mass and solvent and facilitate a reduction in the level of undesirable components remaining in the edible mass. Preferably, agitation does not involve high shear but is preferably arranged not to significantly affect the dimensions of the fungal particles. Thus, a ratio of the average lengths of fungal particles isolated in step (iv) divided by the average lengths of fungal particles in said precursor mass selected in step (i) is at least 0.7, preferably at least 0.9. Said ratio may be about 1.

Suitably, after step (ii), the aqueous solvent entrains components extracted from the fungal particles. Upon filtration in step (iii), the filtrate suitably contains said aqueous solvent and components extracted from the fungal particles. Consequently, the residue which suitably defines the edible mass has a reduced level of certain components (which have been extracted into the aqueous solvent as described). It is found that the components extracted include many of those responsible for the mushroom like taste of the fungal particles.

Consequently, the edible mass produced advantageously has a reduced mushroom like taste/smell.

Said aqueous solvent selected in step (ii), preferably includes at least 70 wt%, preferably at least 95 wt%, more preferably at least 99 wt% water. Said aqueous solvent selected in step (ii), preferably consists essentially of water. It may consist of substantially pure water. It may consist of distilled or deionised water.

A filtrate produced after filtration of said mixture may include one or more components selected from:

| | |
|---|---|
| I | acetyl-glutamic acid; |
| II | uridine; |
| III | inosine; |
| IV | guanosine; |
| V | UMP; |
| VI | GMP; |
| VII | AMP; |
| VIII | sodium; |
| IX | potassium; |
| X | ammonium. |

Said filtrate produced after filtration of said mixture may include at least three, at least seven or all of the components selected from I to X above.

Said filtrate produced after filtration of said mixture may include one or more of components II to VII. Said filtrate produced after filtration of said mixture may include at least three, at least five, or all of components II to VII.

A ratio defined as the total weight of protein contained in the residue divided by the total weight of protein contained in the filtrate is at least 1, is preferably at least 2 and may be at least 5 or at least 10. Thus, the majority of the protein is not extracted into the aqueous solvent and advantageously remains associated with the edible mass. Proteins may be assessed, for example, a spectroscopic Biuret test.

Said precursor mass comprising fungal particles of a filamentous fungus selected in step (i), may be as described in the first aspect, except that amounts of referenced components in the fungal particles may in general be higher (and/or outside the ranges) than described in the first aspect. Said precursor mass may include fungal particles which are non-viable; and/or have levels of RNA; and/or dimensions as described in the first aspect.

In step (b) of the method, said mass may be contacted with water.

In step (b) of the method, said mass may be contacted with other ingredients, for example one or more flavouring materials.

In step (b) of the method, said mass may be contacted with a milk flavour additive.

According to a third aspect, there is provided the use of a precursor mass comprising fungal particles of a filamentous fungus, as described in the second aspect in making an aqueous formulation of the first aspect which has a reduced mushroom like taste and/or flavour.

Any feature of any aspect of any invention described herein may be combined with any feature of any other invention described herein mutatis mutandis.

Specific embodiments of the invention will now be described, by way of example, with reference to the accompanying figures, in which:

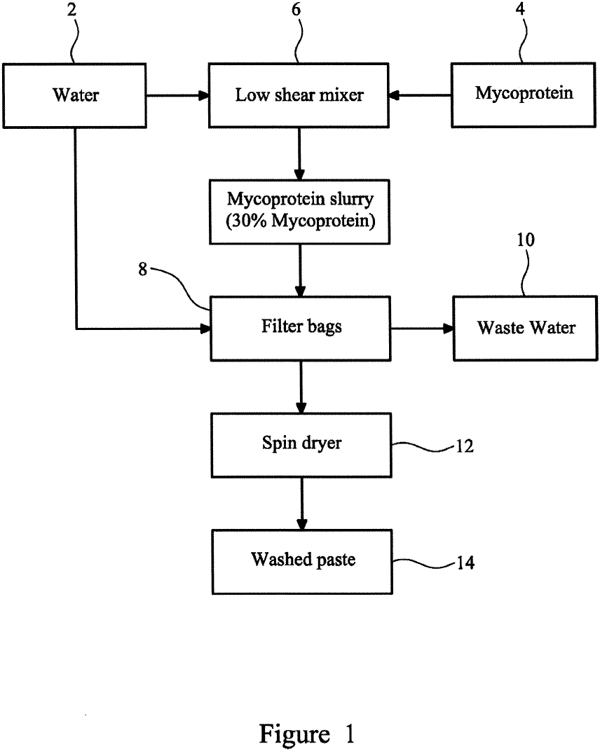

FIG. 1 is a schematic diagram of a first process for washing mycoprotein paste;

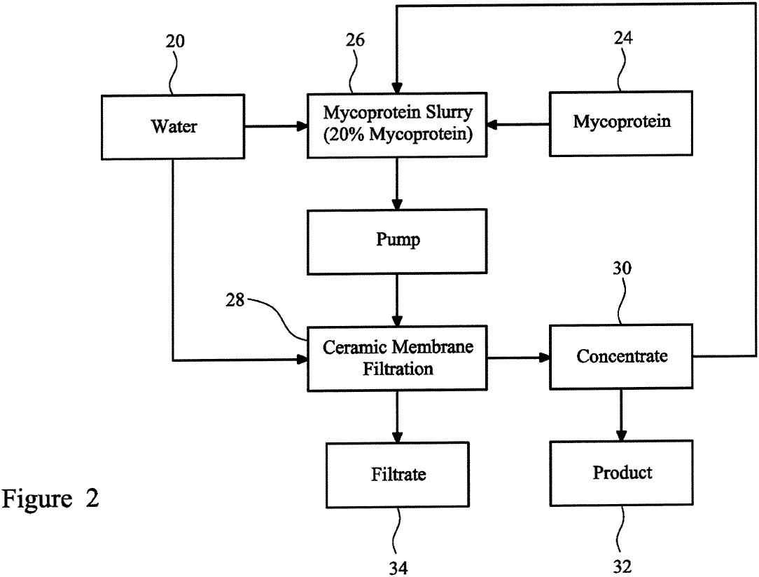

FIG. 2 is a schematic diagram of a second process for washing mycoprotein paste; and

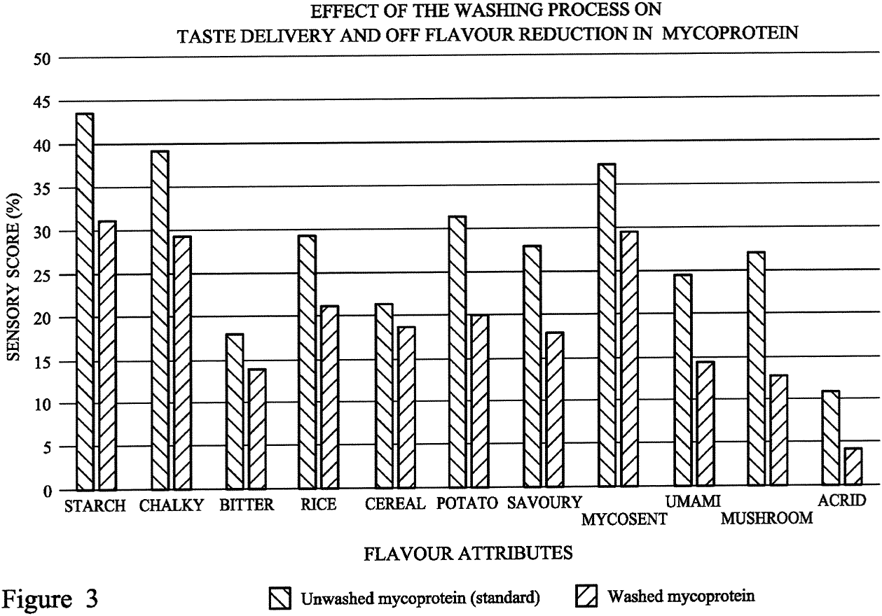

FIG. 3 is a diagram which illustrates the effect of washing on taste as assessed by a trained panel.

The following material is referred to hereinafter:

Mycoprotein paste - Mycoprotein paste-refers to a visco-elastic material comprising a mass of edible filamentous fungus derived from Fusarium venenatum A3/5 (formerly classified as Fusarium graminearum Schwabe) (IMI 145425; ATCC PTA-2684 deposited with the American type Culture Collection, 12301 Parklawn Drive, Rockville Md. 20852) and treated to reduce its RNA content to less than 2% by weight by heat treatment. Further details on the material are provided in WO96/21362 and WO95/23843. The material may be obtained from Marlow Foods Limited of Stokesley, U.K. It comprises about 23-25 wt % solids (the balance being water) made up of non-viable RNA reduced fungal hyphae of approximately 400-750 µm length, 3-5 µm in diameter and a branching frequency of 2-3 tips per hyphal length.

UMP, GMP and AMP refer to uridine monophosphate, guanosine monophosphate and adenosine monophosphate respectively.

The following tests methods are used to analyse mycoprotein paste.

### Test 1 - Analysis of Paste Residual Molecules Before and After Washing

### Material Preparation

A sample of mycoprotein was mixed with water to a ratio of 1 part mycoprotein to 10 parts water. De-ionised water was used for extraction. The mix of sample and water was introduced to a homogenizer (Polytron GT 10-35) and homogenized at high shear at 10-15 k rotations for 1 min. This action produced a slurry. The slurry was filtered through two syringe-filters in a row. The first filter was a Spartan® 30/0.45RC and the second was a Millex GN Nylon 0.2 µm. The supernatant was then then analysed.

### Analysis

### 1. Anions and Organic Acids (Lactate, Acetate, Formate, Chloride, Sulphate, Malate, Succinate, Phosphate, Citrate)

Instrument: High-Performance Ion Chromatography (HPIC). The ICS-3000 ion chromatography system (Dionex, Olten, Switzerland) consisted of two ICS-300 DP pumps (isocratic and gradient), an ICS-3000 autosampler, a DC ICS-3000 thermal compartment, and an amperometric and an electrochemical detector. System control and data acquisition were performed using Chromeleon software (version 6.7, Dionex).

Anions were analyzed using an IonPac AS11-HC analytical column (4 × 250 mm, Dionex) equipped with an lonPac AG11-HC guard column (4 × 50 mm, Dionex) and self-regenerating anion suppressor ASRS-Ultra II (4 mm, Dionex) operating at 223 mA with hydroxide eluent generation. Chromatography was performed at 30° C. with a flow rate of 1.5 mL/min using aqueous potassium hydroxide as solvent and starting with a concentration of 1 mM for 1 min, increasing the ion strength to 30 mM within 14 min, then to 60 mM within 10 min, and maintaining this concentration for 7 min.

### 2. Cations (Sodium, Ammonium, Potassium, Magnesium, Calcium)

Instrument: see Anions. Cations were analyzed using an IonPac CS12-A analytical column (4 × 250 mm, Dionex) equipped with an lonPac CG12-A guard column (4 × 50 mm, Dionex) and self-regenerating cation suppressor CSRS-Ultra II (4 mm, Dionex) operating at 88 mA with methanesulfonic acid eluent generation. Isocratic chromatography was performed at 30° C. with a flow rate of 1.5 mL/min using aqueous methanesulfonic acid with a concentration of 20 mM for 12 min.

### 3. Nucleotides (Adenine, Uridine, Adenosine, Guanosine)

Instrument: High-Performance Liquid Chromatography (HPLC). For analytical HPLC, an Agilent 1100 series HPLC system consisting of a binary pump, an autosampler, a column oven (at 30° C.), an online degasser, and a diode array detector (Agilent, Waldbronn, Germany) was used. Data acquisition was performed using the software HP ChemStation (Agilent, Waldbronn, Germany). Nucleotides were analyzed using an RP-HPLC-DAD method. Therefore, a defined amount of the Quorn was dissolved in deionized water and membrane filtered (0.45 µm). Aliquots were injected onto an RP18 column (Zorbax Eclipse, 150 × 4.6, 5 µm, Agilent, Santa Clara, CA, USA) and separated with a gradient of methanol/acetonitrile (5:4; v/v; solvent A) and 23 mM (NH4)2HPO4 in water (pH 6.0, solvent B). Using a flow rate of 1.0 mL/min, chromatography was started with 100% B; in 25 min the content of A was increased to 30% and maintained for 15 min at that solvent ratio. Detection was performed by means of a DAD set at 254 nm. For quantification six-point external calibration curves were recorded.

### 4. Other Constituents (Homoserine, Pyroglutamic Acid, Strombine, Glutamic Acid)

Instrument: High-Performance Liquid Chromatography-Mass Spectrometry (HPLC-MS/MS). The Agilent 1200 series HPLC system, consisting of a binary pump, an online degasser, a column oven (at 30° C.), and an autosampler (Agilent), was connected to an API 3200 QTRAP mass spectrometer (AB Sciex Instruments, Darmstadt, Germany), which was equipped with an electrospray ionization (ESI) source and operated in the positive ionization mode. The ion spray voltage was set to 3500 or 4000 V depending on the HPLC method (HILIC, 3500 V; PFP, 4000 V), and the declustering potential and the MS/MS parameters were optimized for each substance to induce fragmentation of the pseudo molecular ion [M - H]+ to the corresponding target product ions after collision-induced dissociation. The dwell time for each mass transition was 150 ms, and the declustering potential (DP), the cell exit potential (CXP), and the collision energy (CE) were optimized for each substance. Quantitative analysis was performed by means of the multiple reaction monitoring (MRM) mode using the fragmentation parameters optimized prior to analysis. Data processing and integration were performed by using Analyst software version 1.5.1 (AB Sciex Instruments). For the MRM-IDA-EPI MS experiments an information-dependent acquisition (IDA) method using these MRM survey scans to confirm the presence and identity of the NAG was applied. In the case of the presence of the target analyte, a full scan enhanced product ion (EPI) spectrum of the compound was acquired, and this mass spectrum was compared to the one of the reference compounds. To quantitate some additional taste compounds, two LC-MS/MS multimethods based on two orthogonal HPLC columns (PFP-RP18 and ZIC-HILIC) were developed. A defined amount of the Quorn extract was dissolved in deionized water and membrane filtered (0.20 µm). Aliquots were analyzed on a SeQuant ZIC-HILIC column (150 × 4.6 mm, 5 µm, SeQuant, Umeå, Sweden) and on a Phenomenex Luna PFP column (250 × 4.6 mm, 3 µm, Phenomenex, Aschaffenburg, Germany) via MS/MS. In both cases the target compounds were analyzed by means of HPLC-MS/MS operating in the MRM with positive electrospray ionization.

### Test 2 - Analysis of Protein Content

This was undertaken using a standard method.

### Test 3 - Analysis of Fat Content

This was undertaken using a standard method.

Examples 1 to 3, describe how edible fungal particles with reduced levels of specified components may be prepared.

### Example 1 - Washing of Mycoprotein Paste - First Method (Lab Method)

Referring to FIG. 1, water 2 and mycoprotein paste 4 are contacted and mixed in a low shear mixer 6 to produce a slurry which includes 30 wt% mycoprotein paste. After mixing, the slurry is filtered using filter bags 8 to produce waste water filtrate 10 and solids which are spin dried 12 to produce washed paste 14.

### Example 2 - Washing of Mycoprotein Paste - Second Method (Ceramic Membrane Method

Referring to FIG. 2, water 20 and mycoprotein paste 24 are contacted and mixed and a slurry 26 produced comprising 20 wt% mycoprotein. The slurry is pumped through a ceramic filtration membrane with pore size 0.1 micron at pressures between 4-6 bar, in the presence of additional water. A concentrate 30 is produced and the product 32 comprising washed paste isolated. Waste filtrate 34 is also collected.

### Example 3 - Washing of Mycoprotein Paste - Third Method (Vacuum Belt)

The process of Example 2 is followed except that a vacuum filter belt (7 micron) is used to filter the mycoprotein slurry instead of the ceramic filtration membrane of Example 2,

The washed paste of Examples 1, 2 and/or 3 were analysed as described in Tests 1, 2 and/or 3 and results are provided below.

### Results

Table 1 details results of analyses, following the procedures referred to in Tests 1 to 3 of mycoprotein which has been washed using the processes of Examples 1 to 3. The table also details the amounts of specified compounds/molecules in mycoprotein prior to any washing. The results are quoted based on the amount of mycoprotein on a wet matter basis.

TABLE 1

| Compound/Molecule | Unwashed Mycoprotein (mg/kg paste) | Mycoprotein washed as per Example 1 (mg/kg paste) | Mycoprotein washed as per Example 2 (mg/kg paste) | Mycoprotein washed as per Example 3 (mg/kg paste) |
|---|---|---|---|---|
| UMP | 165.00 | 17.10 | 0.00 | 12.50 |
| GMP | 214.00 | 27.30 | 11.00 | 45.20 |
| AMP | 227.00 | 24.80 | 0.00 | 0.00 |
| Sodium | 81.00 | 76.00 | 77.00 | 112.00 |
| Ammonium | 381.00 | 0.00 | 0.00 | 42.00 |
| Potassium | 564.00 | 129.00 | 166.00 | 264.00 |
| Magnesium | 139.00 | 43.00 | 47.00 | 73.00 |
| Calcium | 107.00 | 40.00 | 60.00 | 66.00 |
| Homoserine | 5.15 | 2.40 | 5.27 | 1.51 |
| Nicotineamide | 0.48 | 0.68 | 0.00 | 0.00 |
| Pyroglutamic acid | 4.93 | 1.87 | 0.58 | 2.17 |
| Hypoxanthine | 16.28 | 0.73 | 4.70 | 25.11 |
| Uridine | 42.30 | 4.94 | 12.10 | 19.75 |
| Adenosine | 37.08 | 8.68 | 7.57 | 0.56 |
| Inosine | 18.69 | 5.48 | 7.40 | 8.07 |
| Guanosine | 158.76 | 16.22 | 40.20 | 25.95 |
| Acetyglutamic acid | 0.79 | 0.09 | 0.20 | 0.39 |

As a result of Applicant’s assessment, it is believed certain compounds/molecules contribute most significantly to the mushroom like flavour of the mycoprotein which it is desired to reduce. Table 2 details such compounds/molecules contained in unwashed mycoprotein and in mycoprotein washed as described in Example 1. The results are quoted based on the amount of mycoprotein on a wet matter basis.

TABLE 2

| Compound/Molecule/lon | Unwashed Mycoprotein (mg/kg) | Mycoprotein washed as per Example 1 |
|---|---|---|
| UMP | 165.00 | 17.10 |
| GMP | 214.00 | 27.30 |
| AMP | 227.00 | 24.80 |
| Ammonium ions | 381.00 | 0.00 |
| Potassium ions | 564.00 | 129.00 |
| Uridine | 42.30 | 4.94 |
| Inosine | 18.69 | 5.48 |
| Guanosine | 158.76 | 16.22 |
| Acetylglutamic acid | 0.79 | 0.09 |

Mycoprotein is used as a foodstuff and, more particularly, as a source of dietary protein. Consequently, it is desirable that any treatment does not reduce the amount of amino acids/protein within the mycoprotein after the treatments described. Table 3 details results of analysis of the levels of certain amino acids in unwashed mycoprotein and in mycoprotein washed as described in Example 1. The results are quoted based on the amount of mycoprotein on a wet matter basis.

TABLE 3

| Analysis | Unwashed Mycoprotein | Mycoprotein washed as per Example 1 |
|---|---|---|
| Aspartic Acid (g/100 g) | 1.36 | 1.47 |
| Serine (g/100 g) | 0.65 | 0.70 |
| Glutamic Acid (g/100 g) | 1.67 | 1.72 |
| Glycine (g/100 g) | 0.65 | 0.69 |
| Histidine (g/100 g) | 0.33 | 0.36 |
| Arginine (g/100 g) | 0.93 | 1.01 |
| Threonine (g/100 g) | 0.71 | 0.77 |
| Alanine (g/100 g) | 0.87 | 0.90 |
| Proline (g/100 g) | 0.60 | 0.64 |
| Cystine (g/100 g) | 0.09 | 0.10 |
| Tyrosine (g/100 g) | 0.38 | 0.41 |
| Valine (g/100 g) | 0.83 | 0.92 |
| Methionine (g/100 g) | 0.25 | 0.27 |
| Lysine (g/100 g) | 1.12 | 1.21 |
| Iso-Leucine (g/100 g) | 0.67 | 0.73 |
| Leucine (g/100 g) | 0.99 | 1.07 |
| Phenylalanine (g/100 g) | 0.51 | 0.56 |
| Tryptophan (g/100 g) | 0.20 | 0.22 |

It has been found that the processes described do not significantly detrimentally affect the nutritional value of the mycoprotein - the mycoprotein is found, after washing as described, to be calorifically almost identical to the unwashed mycoprotein and to include levels of fat, carbohydrate and protein comparable with the unwashed mycoprotein.

Mycoprotein treated as described in Example 1 was assessed for taste delivery and off-flavour reduction by a trained panel of individuals who assessed a range of flavour attributes. Results are provided in FIG. 3, wherein the unwashed (standard mycoprotein) is shown on the left and the washed mycoprotein is shown on the right for each attribute. It should be noted that, for each of the attributes assessed, the savoury score is reduced, meaning that the particular flavour attribute is reduced in the washed protein.

Advantageously, mycoprotein which has been washed as described has a less savoury and/or mushroom like flavour. Consequently, it may be used in foodstuffs where it is desired to minimise such flavours However, other important nutritional characteristics, such as amino acids and proteins are not detrimentally reduced.

The following example illustrates how a milk may be produced.

### Example 4 - Preparation of Milk-Like Liquid Foodstuff

Mycoprotein paste which had been washed as described in Example 1 was selected. 25 wt% of the paste (which itself contains approximately 19 wt% water) was added to water (75 wt%) and the mixture microfluidised to produce a milk base. Such a treatment significantly reduces hyphal lengths and viscosity of the fluid.

Thereafter, the following ingredients were mixed into the mixture at the levels indicated in the table below (which also details the amount of milk base).

| Ingredients | wt% |
|---|---|
| sugar | 0.10 |
| salt | 0.08 |
| soy soother | 0.02 |
| milk flavour | 0.01 |
| pectin | 0.35 |
| milk base | 99.44 |

The milk base includes about 5.7% of mycoprotein on a dry matter basis.

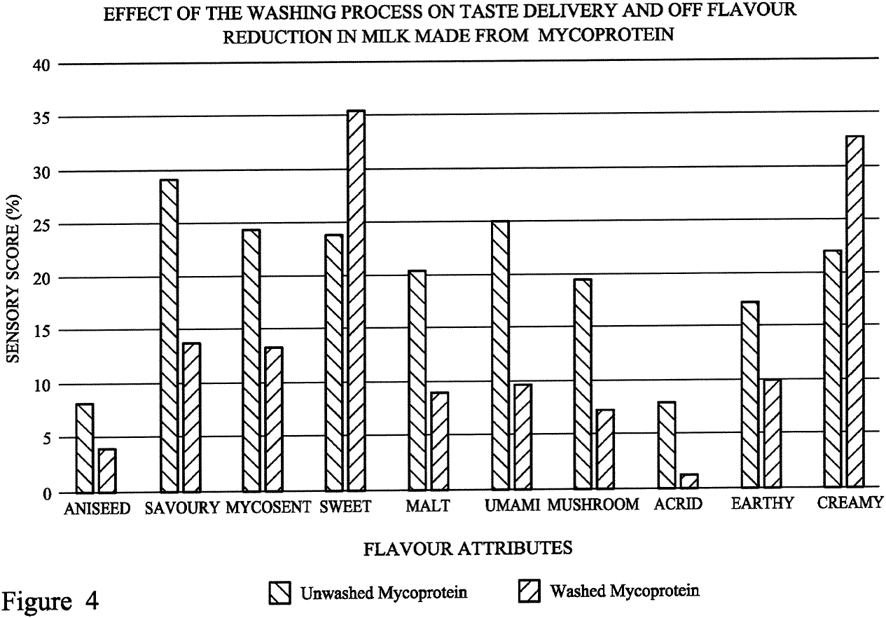

The liquid foodstuff of Example 4 made using the washed paste was assessed and compared to an otherwise identical liquid foodstuff (referred to as Example C1) prepared from virgin paste which had not been washed, for example as described in Examples 1 to 3. A trained panel assessed the foodstuffs of Examples 4 and C1 using predetermined criteria and results are provided in FIG. 4, wherein the unwashed (standard mycoprotein) is shown on the left and the washed mycoprotein is shown on the right for each attribute.

Referring to FIG. 4, the following should be noted:
the Example 4 foodstuff has reduced aniseed flavour compared to Example C1;
the Example 4 foodstuff has reduced savoury flavour compared to Example C1;
the Example 4 foodstuff has an increased sweetness compared to Example C1. In light of this, the amount of sugar which may need to be included in the liquid foodstuff to achieve consumer acceptability may advantageously be less than for a foodstuff based on Example C1;
the Example 4 foodstuff has reduced malt flavour compared to Example C1;
the Example 4 foodstuff has reduced umami (meaty) flavour compared to Example C1;
the Example 4 foodstuff has reduced mushroom like flavour compared to Example C1;
the Example 4 foodstuff has reduced acrid flavour compared to Example C1;
the Example 4 foodstuff has reduced earthy flavour compared to Example C1;
the Example 4 foodstuff advantageously has an increased creamy flavour compared to Example C1.

### Example 5 - Nutritional Assessment of Liquid Formulation of Example 4

The liquid formulation of Example 4 was assessed for its protein content and its fat content by conventional techniques and the results are provided in the following tables which also include comparative values for 1% fat cow’s milk and for commercially available soy milk.

| Product | Protein content (g/100 ml) | Fat content (g/100 ml) |
|---|---|---|
| Example 4 formulation | 2.8 | 0.75 |
| 1% fat cow’s milk | 3.4 | 1.0 |
| soy milk | 3.3 | 1.8 |

Thus, surprisingly and unpredictably, the Example 4 foodstuff shows improvements in desirable flavours (e.g. sweet and creamy flavours) whilst reducing undesirable savoury, meat and/or mushroom like flavours, as illustrated in FIG. 4. In addition, the process described may minimise loss of biomass and hence the amino acids/proteins and be used to produce liquid formulations which deliver high levels of amino acids/proteins. The liquid formulations may therefore advantageously be used to deliver a high level anabolic response in a consumer-acceptable manner.

### Example 6 - Preparation of Alternative Milk-Like Liquid Foodstuff

An alternative recipe for a milk-like liquid foodstuff which produces an excellent product is as follows:

| Ingredient | wt% |
|---|---|
| Milk base | 99.5 |
| Sugar | 0.1 |
| Salt | 0.1 |
| Gellan gum | 0.1 |
| Natural flavour | 0.2 |
| | 100.0 |

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

1. An aqueous formulation comprising fungal particles of a filamentous fungus.

2. A formulation according to claim 1, wherein said fungal particles collectively include at least one of the following parts per million of a specified component described in (a) to (j), relative to the total mass of the fungal particles, on a dry matter basis:
(a)less than 3 ppm of acetyl-glutamic acid;
(b) less than 160 ppm of uridine;
(c) less than 75 ppm of inosine;
(d)less than 625 ppm of guanosine;
(e)less than 660 ppm of UMP;
(f) less than 800 ppm of GMP;
(g)less than 875 ppm of AMP;
(h)less than 330 ppm of sodium;
(i) less than 2300 ppm of potassium;
(j) less than 1500 ppm of ammonium.

3. A formulation according to claim 2, wherein said fungal particles include at least six of the characteristics (a) to (j).

4. A formulation according to claim 1, wherein the fungal particles collectively include at least six of the following parts per million of a specified component described in (k) to (t), relative to the total mass of the fungal particles, on a dry matter basis:
(k) less than 2 ppm of acetyl-glutamic acid;
(l) less than 80 ppm of uridine;
(m) less than 50 ppm of inosine;
(n) less than 400 ppm of guanosine;
(o) less than 400 ppm of UMP;
(p) less than 400 ppm of GMP;
(q) less than 400 ppm of AMP;
(r) less than 330 ppm of sodium;
(s) less than 1000 ppm of potassium;
(t) ess than 1000 ppm of ammonium.

5. (canceled)

6. (canceled)

7. A formulation according to claim 1, wherein said fungal particles include at least one of the following characteristics, wherein an amount of a component is specified per 100 g of said fungal particles on a dry matter basis:
(A) at least 4.1 g/100 g of aspartic acid;
(B) at least 2.0 g/100 g of serine;
(C) at least 6.0 g/100 g of glutamic acid;
(D) at least 2.0 g/100 g of glycine;
(E) at least 1.0 g/100 g of histidine;
(F) at least 2.8 g/100 g of arginine;
(G) at least 2.0 g/100 g of threonine;
(H) at least 0.6 g/100 g of alanine;
(I) at least 2.4 g/100 g of proline;
(J) at least 0.2 g/100 g of cystine;
(K) at least 1.2 g/100 g of tyrosine;
(L) at least 2.8 g/100 g of valine;
(M) at least 0.8 g/100 g of methionine;
(N) at least 4.0 g/100 g of lysine;
(O) at least 2.0 g/100 g of iso-leucine;
(P) at least 3.2 g/100 g of leucine
(Q) at least 1.6 g/100 g of phenylalanine;
(R) at least 0.6 g/100 g of tryptophan.

8. A formulation according to claim 7, wherein said fungal particles include at least five of characteristics (A) to (R).

9. A formulation according to claim 2, wherein said fungal particles comprise fungus selected from Fusarium species.

10. (canceled)

11. (canceled)

12. A formulation according to claim 3, wherein said aqueous formulation comprises at least 5 wt% of said fungal particles on a dry matter basis; and wherein said aqueous formulation comprises at least 80 wt% water.

13. (canceled)

14. A formulation according to claim 12, wherein in said aqueous formulation, the ratio defined as wt% of water divided by the wt% of fungal particles, [(]said fungal particles being on a dry matter basis[)], is in the range 3 to 6; and wherein in said aqueous formulation, the sum of the wt% of fungal particles and water is at least 90 wt%.

15. (canceled)

16. A formulation according to claim 3, wherein said aqueous formulation is a milk.

17. A formulation according to claim 16, wherein said aqueous formulation incudes 0 wt% of ingredients derived from an animal source.

18. A formulation according to claim 17, wherein said aqueous formulation incorporates a milk flavour additive.

19. A method of making an aqueous formulation according to claim 1, the method comprising:
(a) selecting an edible mass comprising fungal particles of a filamentous fungus; and
(b) contacting said mass with one or more other ingredients.

20. A method according to claim 19, wherein said edible mass comprising fungal particles of a filamentous fungus is made in a method comprising:
(i) selecting a precursor mass comprising fungal particles of a filamentous fungus;
(ii) contacting said precursor mass with an aqueous solvent to produce a mixture;
(iii) filtering said mixture;
(iv) isolating a residue which comprises a said edible mass comprising fungal particles of a filamentous fungus.

21. A method according to claim 20, wherein in step (b), said mass is contacted with water.

22. (canceled)

23. A method according to claim 21, wherein[,] in step (b), said mass is contacted with a milk flavour additive.

24. (canceled)

25. A method according to claim 20, wherein the method is a method of making an aqueous formulation which has a reduced mushroom-like taste and/or flavour and has less than 660 ppm of UMP; less than 800 ppm of GMP; and less than 875 ppm of AMP.

26. A formulation according to claim 3, wherein said aqueous formulation comprises:
at least 17.5 wt% of said fungal particles on a dry matter basis;
at least 80 wt% water;
a ratio defined as wt% of water divided by the wt% of fungal particles, said fungal particles being on a dry matter basis, being in the range 4 to 5; and
wherein in said aqueous formulation, the sum of the wt% of fungal particles and water is at least 90 wt%.

27. An aqueous formulation comprising fungal particles of a filamentous fungus, wherein said fungal particles collectively include at least six of the following parts per million of a specified component relative to the total mass of the fungal particles, on a dry matter basis:
(u) less than 3 ppm of acetyl-glulamic acid;
(v) less than 160 ppm of uridine;
(w) less than 75 ppm of inosine;
(x) less than 625 ppm of quanosine;
(y) less than 660 ppm of UMP;
(z) less than 800 ppm of GMP;
(aa) less than 875 ppm of AMP;
(bb) less than 330 ppm of sodium;
(cc) less than 2300 ppm of potassium;
(dd) less than 1500 ppm of ammonium; and
wherein said fungal particles include at least five of the following characteristics, wherein an amount of a component is specified per 100 g of said fungal particles on a dry matter basis:
(S) at least 4.1 g/100 g of aspartic acid;
(T) at least 2.0 g/100 g of serine;
(U) at least 6.0 g/100 g of glutamic acid;
(V) at least 2.0 g/100 g of glycine;
(W) at least 1.0 g/100 g of histidine;
(X) at least 2.8 g/100 g of arginine;
(Y) at least 2.0 g/100 g of threonine;
(Z) at least 0.6 g/100 g of alanine;
(AA) at least 2.4 g/100 g of proline;
(BB) at least 0.2 g/100 g of cystine;
(CC) at least 1.2 g/100 g of tyrosine;
(DD) at least 2.8 g/100 g of valine;
(EE) at least 0.8 g/100 g of methionine;
(FF) at least 4.0 g/100 g of lysine;
(GG) at least 2.0 g/100 g of iso-leucine;
(HH) at least 3.2 g/100 g of leucine;
(II) at least 1.6 g/100 g of phenylalanine;
(JJ) at least 0.6 g/100 g of tryptophan.

28. A formulation according to claim 27, wherein:
said fungal particles comprise cells of Fusarium species;
said aqueous formulation comprises at least 5 wt% of said fungal particles on a dry matter basis;
said aqueous formulation comprises at least 80 wt% water;
in said aqueous formulation, the ratio defined as wt% of water divided by the wt% of fungal particles, said fungal particles being on a dry matter basis, is in the range of 3 to 6;
in said aqueous formulation, the sum of the wt% of fungal particles and water is at least 90wt%; and
said aqueous formulation is a milk.
